(19) United States
(12) Patent Application Publication
(10) Pub. No.: US 2026/0276513 A1
(43) Pub. Date: Sep. 17, 2026

(54) A CELL COUNTER AND DIAGNOSTIC DEVICE

(71) Applicant: MICROBIOSENSOR LIMITED, Manchester (GB)

(72) Inventors: Michael Gordon BARKER, Manchester (GB); Curtis DOBSON, Manchester (GB); Nishal GOVINDJI-BHATT, Manchester (GB); Darren KELL, Manchester (GB); Nicholas GODDARD, Manchester (GB)

(21) Appl. No.: 19/470,511

(22) PCT Filed: Mar. 28, 2024
(86) PCT No.: PCT/GB2024/050854

§ 371 Date: Sep. 29, 2025

(30) Foreign Application Priority Data
Mar. 31, 2023 (GB) 2304863.0

(51) Int. Cl. G01N 15/075; A61B 5/00; A61B 5/145; A61B 5/1455; A61M 1/14; A61M 1/28; G01N 15/01

(52) U.S. Cl. CPC G01N 15/075; A61B 5/14546; A61B 5/1455; A61B 5/6866; A61M 1/154; A61M 1/282; G01N 15/01

(57) ABSTRACT

A cell counter and diagnostic device Disclosed is a device for determining a concentration of cells of a given size in a sample of 5 fluid, the sample comprising a plurality of cells, comprising: a light source arranged to emit light along a central axis to illuminate the plurality of cells in the fluid sample; a container to contain the sample; a detector arranged to receive light at a plurality of displacements from the central axis, the light having passed through the plurality of cells in the fluid sample, and to produce a plurality of signals, each associated with, and indicative of an intensity of the received light at, 10 a respective one of the plurality of displacements; and a processor arranged to: obtain a first light intensity reading at a first displacement from the central axis; obtain a second light intensity reading at a second displacement from the central axis; determine a difference between the first and second light intensity readings; determine the concentration of cells based on the difference; wherein the container is one of: an effluent line arranged to convey 15 effluent from a patient; an effluent bag arranged to collect effluent from a patient; and a cassette forming part of an automated peritoneal dialysis system. FIG. 9

The present invention relates to a cell counter and diagnostic device able to be used in a variety of situations, including accompanying treatments such as peritoneal dialysis and laboratory work, where determining the concentration of cells suspended in a medium or physiological liquid or solution is required.

Peritoneal dialysis is a treatment used with human patients where the patient's kidneys are no longer able to perform adequately. The treatment assists in filtering unwanted waste products from the patient's blood. Peritoneal dialysis is used as an alternative to haemodialysis. In some instances, peritoneal dialysis is preferred since it can be performed without medical supervision and in the patient's home. It may also find utility in assisting in the diagnosing of spontaneous bacterial peritonitis in patients with ascites

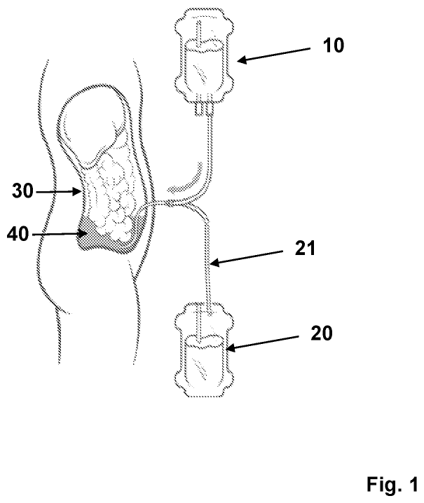

FIG. 1 shows a schematic view of a typical peritoneal dialysis set up. The dialysis solution 10 is introduced into the patient's peritoneal cavity 40, via a port, whereby the patient's peritoneal lining 30 acts to filter the patient's blood. The waste effluent fluid 20 is collected for disposal.

A problem with such an arrangement is that it is possible for microbes in the peritoneal cavity to cause peritonitis, which can have life threatening consequences for the patient. It is not currently easy to monitor the levels of microbes (e.g. bacteria or fungi) in the peritoneal cavity and it is an aim of an embodiment of the present invention to address this issue and other issues not outlined here.

In the case of laboratory work, there are various techniques available for determining a cell concentration in a sample. However, many of these are complex and require relatively expensive equipment (e.g. cytometry). It is an aim of an embodiment of the present invention to address shortcomings with such prior art solutions and provide alternative devices.

According to the present invention there is provided an apparatus and method as set forth in the appended claims. Other features of the invention will be apparent from the dependent claims, and the description which follows.

According to an aspect of the present invention, there is provided a device for determining a concentration of cells of a given size in a sample of fluid, the sample comprising a plurality of cells, comprising: a light source arranged to emit light along a central axis to illuminate the plurality of cells in the fluid sample; a container to contain the sample; a detector arranged to receive light at a plurality of displacements from the central axis, the light having passed through the plurality of cells in the fluid sample, and to produce a plurality of signals, each associated with, and indicative of an intensity of the received light at, a respective one of the plurality of displacements; and a processor arranged to: obtain a first light intensity reading at a first displacement from the central axis; obtain a second light intensity reading at a second displacement from the central axis; determine a difference between the first and second light intensity readings; determine the concentration of cells based on the difference; wherein the container is one of: an effluent line arranged to convey effluent from a patient; an effluent bag arranged to collect effluent from a patient; and a cassette forming part of an automated peritoneal dialysis system.

In an embodiment, if the container is the effluent line, optical properties of the effluent line are utilised in a light path from the light source to the detector.

In an embodiment, the optical properties of the effluent line include focussing or collimating.

In an embodiment, the container is an effluent line arranged to convey effluent from a patient, wherein collimated light from the light source is focused by a first lens, such that it focuses within the effluent line.

In an embodiment, the focused light diverges beyond the focal point and is then collimated and focussed on the detector by a second lens arrangement.

In an embodiment, the second lens arrangement comprises either:
a pair of lenses where the first of the pair acts to collimate and the second of the pair acts to focus the collimated light on the detector; or
a biconvex lens.

In an embodiment, the container is an effluent line arranged to convey effluent from a patient and the device comprises a receiver for receiving and temporarily retaining the effluent line in a configuration wherein the effluent line is accommodated between the light source and the detector.

In an embodiment, the receiver is a channel dimensioned to closely match the dimensions of the effluent line such that the effluent line is held securely in position inserting the effluent into the channel.

In an embodiment, if the container is the effluent bag, the device is arranged to receive the effluent bag such that the light source and detector are position on opposed sides of the effluent bag such that a light path therebetween passes through the effluent bag.

In an embodiment, the device comprises a receiver for the effluent bag and a lid, arranged such that closure of the lid positions the light source and detector in alignment with the effluent bag positioned therebetween.

In an embodiment, the lid comprises means for flattening a proximal surface of the effluent bag in the vicinity of the light source.

In an embodiment, the means for flattening comprises a pair of elliptical rollers, each arranged to rotate about a respective axis, whereby closure of the lid causes the rollers to engage with the proximal surface of the effluent bag, thereby flattening it.

In an embodiment, if the container is the cassette, the light source and detector are arranged to be positioned, in use, on either side of the cassette, such that a light path therebetween passes through a selected area of the cassette.

In an embodiment, the device is a diagnostic device.

In an embodiment, the detector comprises a plurality of photodetectors, each associated with one of the plurality of displacements.

In an embodiment, the plurality of photodetectors are arranged on both sides of the central axis, such that a first subset of the plurality of displacements is positioned on a first side of the central axis and a second, non-overlapping, subset of the plurality of displacements is positioned on a second side of the central axis.

In an embodiment, the light source is arranged to emit light of a single wavelength.

In an embodiment, the light source is a laser.

In an embodiment, the single wavelength of light is in the range of 650 nm to 700 nm.

According to a further aspect of the invention, there is provided a device for determining a concentration of cells of a given size in a sample of fluid, the sample comprising a plurality of cells, comprising: a light source arranged to emit light along a central axis to illuminate the plurality of cells in the fluid sample; a detector arranged to receive light at a plurality of displacements from the central axis, the light having passed through the plurality of cells in the fluid sample, and to produce a plurality of signals, each associated with, and indicative of an intensity of the received light at, a respective one of the plurality of displacements; and a processor arranged to determine a concentration of cells of a given size, based on the plurality of signals from the detector, wherein the processor is arranged to compare the plurality of signals from the detector with one or more pre-stored thresholds or profiles, indicative of a particular concentration.

In an embodiment, the processor is arranged to: measure a first light intensity reading at a first displacement from the central axis and measure a second light intensity reading at a second displacement from the central axis and determine, using a first method, the concentration of cells on the basis of the first and second light intensity readings and a difference therebetween.

In an embodiment, if the difference between the first and second light intensity is less than a predefined threshold, then the processor is arranged to determine the concentration of cells using a second method In an embodiment, the first and second light intensity readings are taken at displacements proximal the central axis.

In an embodiment, the plurality of displacements are in the range of substantially 1° to 15°, measured from the central axis.

In an embodiment, the detector comprises a plurality of photodetectors, each associated with one of the plurality of displacements.

In an embodiment, the plurality of photodetectors are arranged on both sides of the central axis, such that a first subset of the plurality of displacements is positioned on a first side of the central axis and a second, non-overlapping, subset of the plurality of displacements is positioned on a second side of the central axis.

In an embodiment, the first method uses an Area Under the Curve, AUC, method and the second method uses a Generalised Weighted Ratio, GWR, method.

In an embodiment, the sample is contained within a container, wherein the container either contains a static sample or a dynamic, flowing, sample.

In an embodiment, the container is arranged such that light from the light source strikes it an angle sufficient to reduce reflection of light back to the light source.

In an embodiment, the device is arranged to provide user feedback in the form of one or more of a numerical concentration value or a warning that the concentration value exceeds a predefined threshold.

In an embodiment, the user feedback additionally comprises one or more of: an audible signal to the user; a visual signal to the user; and communicating with a remote device to transmit the concentration of cells to the remote device.

According to a second aspect of the invention, there is provided a container for use with the device of the first aspect, comprising an entry and an exit, defining respective ends of a flow path, whereby there is provided proximal the entry and the exit a kinked portion arranged to minimise light transmission into the container.

In an embodiment, the cuvette has a male connector at a first end and a female connector at a second end to couple to an effluent line.

According to a third aspect of the invention, there is provided a container for use with the device of the first aspect wherein a pair of windows is provided permitting light to enter and exit the container, whereby the pair of windows are recessed from an outer surface such that contact from a user's fingers is prevented or inhibited.

In an embodiment, an exterior of the surface of the container is opaque, except for the pair of windows.

In an embodiment, the container is keyed such that it may only be inserted in one orientation to the device of the first aspect.

Although a few preferred embodiments of the present invention have been shown and described, it will be appreciated by those skilled in the art that various changes and modifications might be made without departing from the scope of the invention, as defined in the appended claims.

For a better understanding of the invention, and to show how embodiments of the same may be carried into effect, reference will now be made, by way of example only, to the accompanying diagrammatic drawings in which:

FIG. 1 shows a peritoneal dialysis setup known in the prior art;

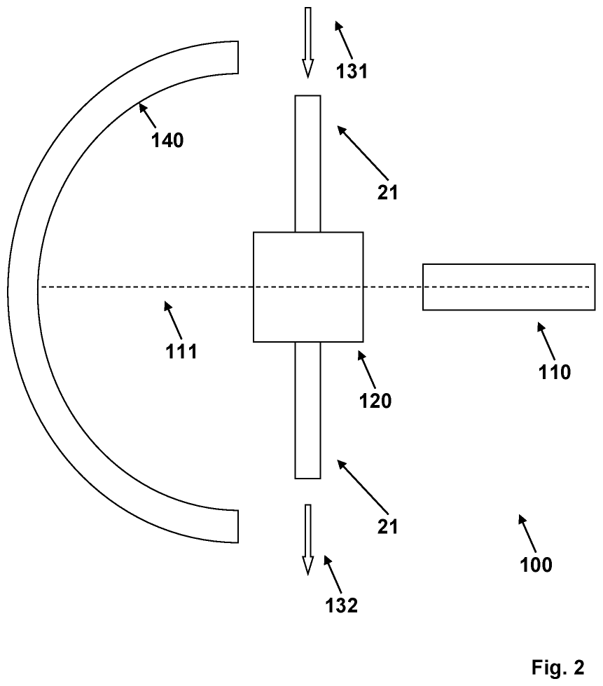

FIG. 2 shows a schematic representation of an embodiment of the present invention;

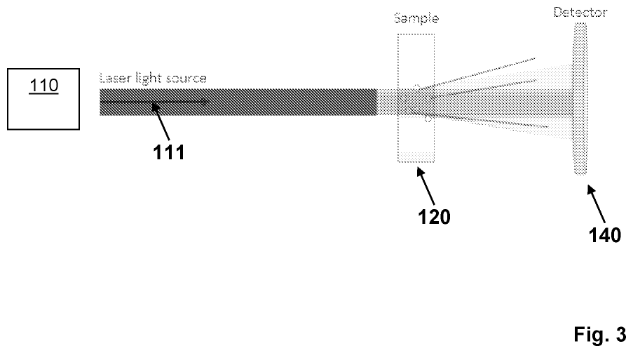

FIG. 3 shows a representation of light scattering which underlies embodiments of the invention;

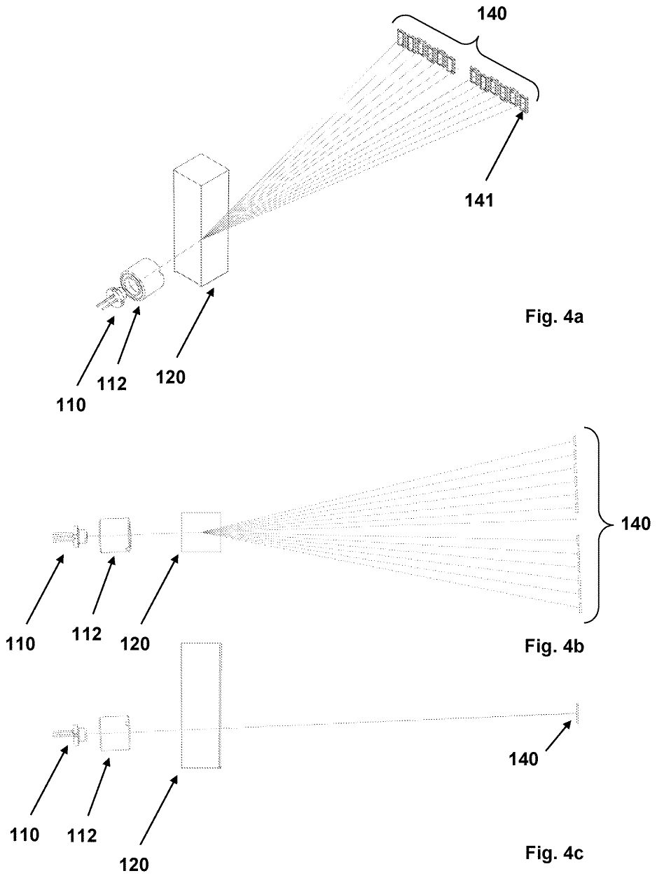

FIGS. 4a-c show details of the arrangement of a cuvette or container, optical source and detectors according to an embodiment of the present invention;

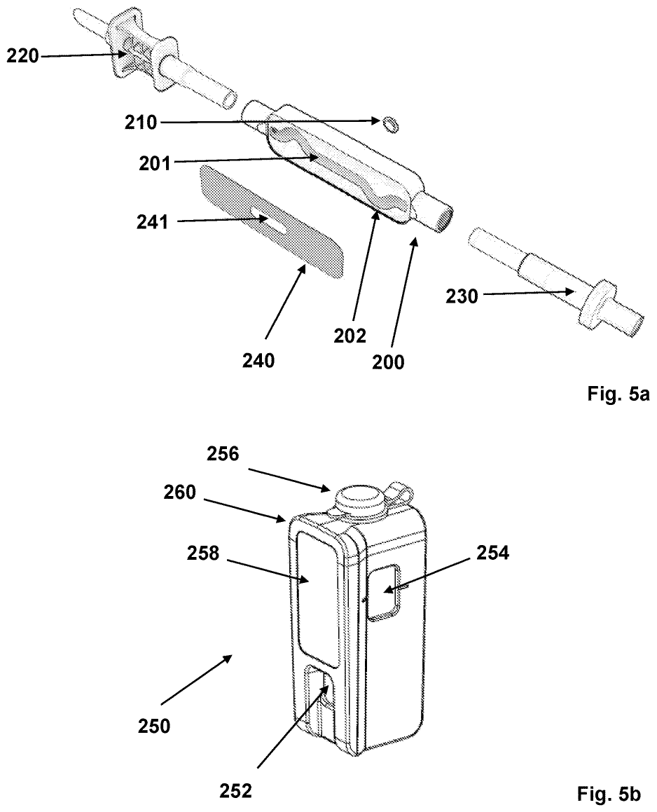

FIG. 5a shows a cuvette according to an embodiment of the present invention;

FIG. 5b shows another cuvette according to an embodiment of the present invention;

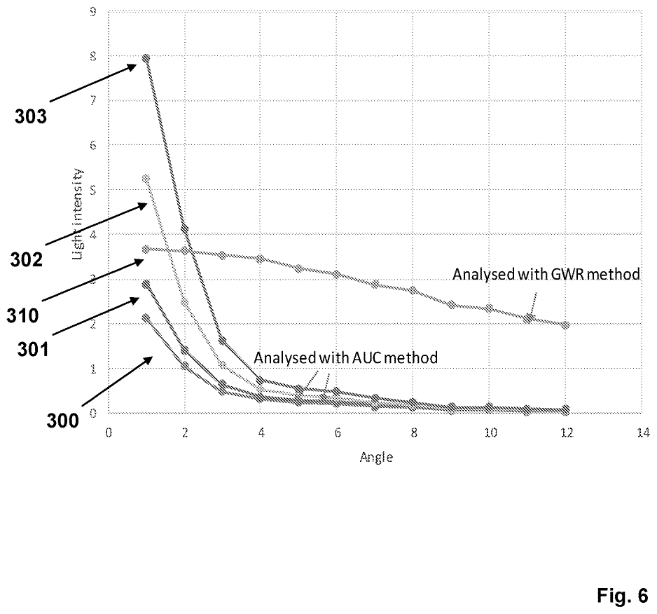

FIG. 6 shows a graph illustrating responses according to different concentrations of leukocytes in a sample;

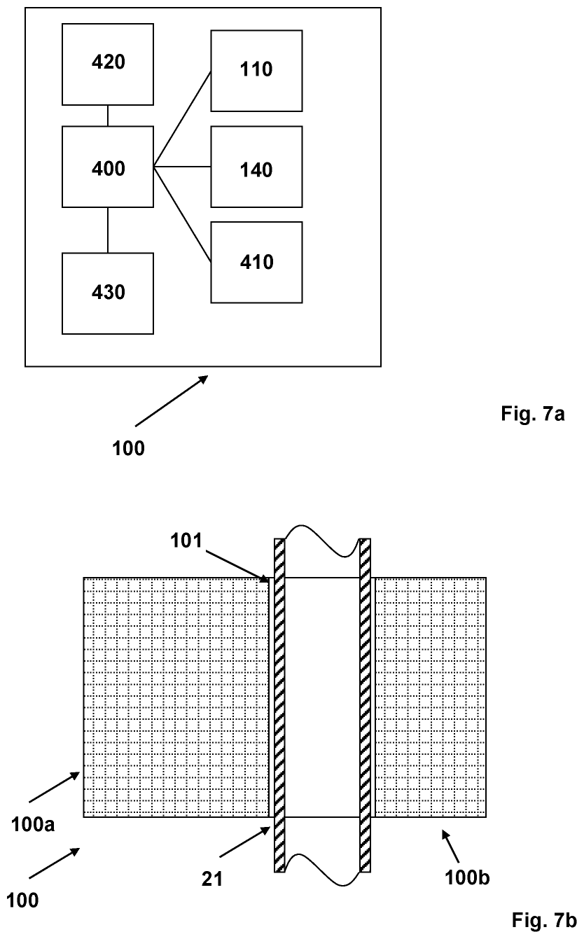

FIG. 7a shows a schematic of certain functional parts of a device according to an embodiment of the invention;

FIG. 7b shows an illustration of a physical embodiment of a device according to an embodiment of the invention;

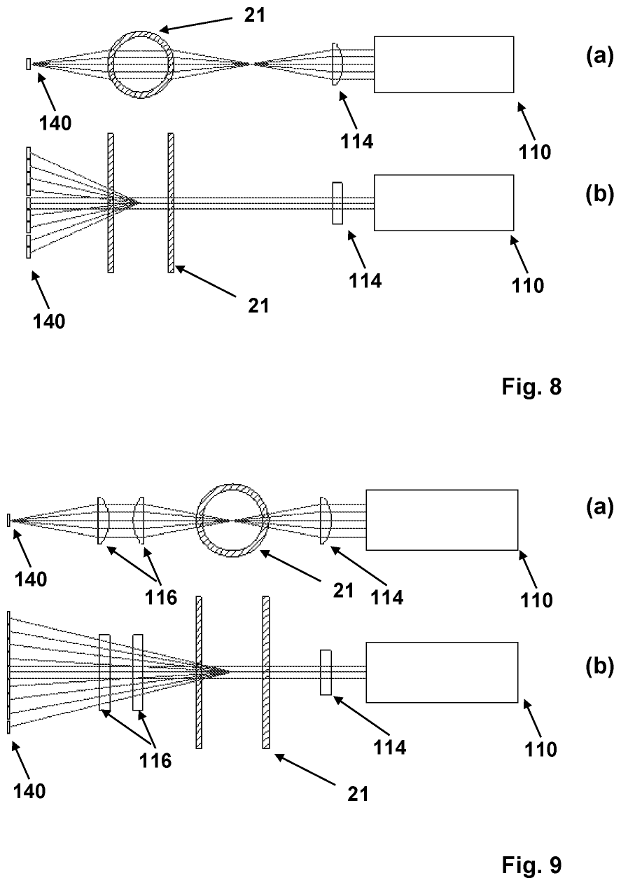

FIG. 8 shows detail of an embodiment providing a first option for beam focussing;

FIG. 9 shows detail of an embodiment providing a second option for beam focussing;

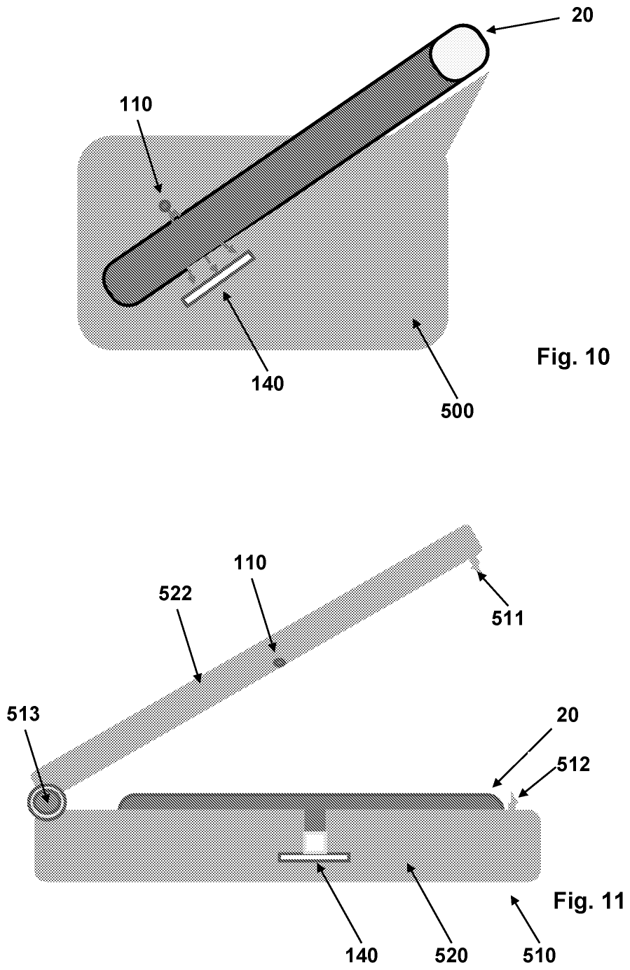

FIG. 10 shows a representative view of an embodiment for analysing a sample in-situ in an effluent reservoir;

FIGS. 11-15 show representative views of a further embodiment for analysing a sample in-situ in an effluent reservoir;

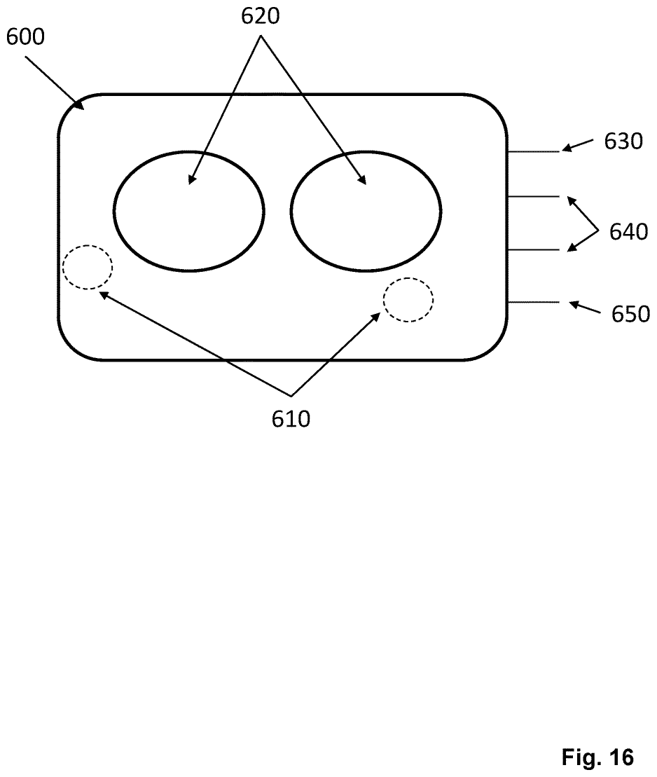

FIG. 16 shows a peritoneal dialysis cassette with certain areas indicated, suitable for effluent analysis by an embodiment of the invention; and

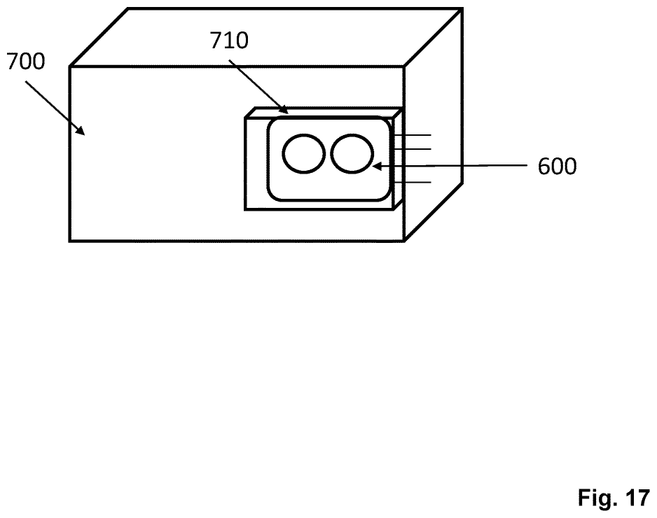

FIG. 17 shows an Automated Peritoneal Dialysis device, with a cassette in situ.

Embodiments of the present invention operate to detect the presence of cells in the effluent fluid 20 during peritoneal dialysis. The cells in question are typically leukocytes. A subset of leukocytes includes neutrophils. Neutrophils above a certain concentration are indicative of the patient fighting an infection. If left unchecked, such an infection can develop into a serious, potentially life threatening, occurrence of peritonitis. Early detection of infection, by the indirect detection of a certain concentration of neutrophils, can be lifesaving. At the very least, it can provide an opportunity for the patient to be treated early, thereby avoiding possible hospital admission and the associated costs and risks of complication. If a bacterial infection is detected, then prompt treatment by a suitable antibiotic can be arranged.

FIG. 2 shows a representation of the mode of operation of a first embodiment of the present invention. This is not intended to represent the actual construction of the device and is to illustrate a mode of operation only. The device 100 is arranged to be attached to the effluent waste pipe 21, such that effluent from the peritoneal dialysis process flows through the device 100. Effluent flows in at 131 and flows out at 132 into the effluent reservoir 20.

Interposed between entry port 131 and exit port 132 is a sample-receiving effluent reservoir, container or cuvette 120 which temporarily holds a sample of effluent fluid for analysis. In another embodiment, there is no flow of effluent and a static sample is analysed instead. All other operational details are the same.

In the first (dynamic) embodiment, a flowing sample of effluent (or other fluid) is analysed whilst flowing to the effluent reservoir 20 or other receptacle. In this case, the fluid flows through the analysis device en-route to the reservoir 20 and so the user is not required to dispose of any sample after analysis.

In contrast, in a second (static) embodiment, the sample may be introduced into the cuvette or other suitable receptacle by means of a syringe or similar, whereby the sample is drawn off from or en-route to the effluent reservoir 20. In this case, the sample may be analysed and then disposed of. The second embodiment may be substantially similar to the first embodiment, except that there is no continuous flow of effluent 131, 132. Alternatively, a different form of receptacle may be employed.

One advantage of embodiments of the present invention is that in both the first and second embodiments, the sample for analysis is not required to undergo any preparatory treatment and so embodiments of the invention are well suited for use at home and/or by unskilled persons.

The sample in the cuvette 120, whether static or dynamic, is illuminated via a laser 110. In one embodiment, the laser is a 5 mW red laser having a wavelength in the region of 650-700 nm. In a preferred embodiment, the LASER is provided in the form of a LASER diode. However, different embodiments may employ different powers and/or wavelengths, as required. In order to achieve the desired effect, a collimating system, such as a lens, may also be required.

Light from the laser or LED 110 passes through the cuvette 120, including the sample, and is detected by a detector 140 located at an opposite side of the cuvette 120. The detector 140 comprises one or more photodiodes which is/are sensitive to light at or about the wavelength transmitted by the laser or LED 110. The detector produces an electrical signal indicative of the strength of the received light. The electrical signal also provides position/angular information indicative of the angular displacement from a central axis 111. In this way, the intensity of light at one or more displacements can be analysed.

The detector is arranged to be exposed to light scattered through the sample. One way to achieve this is to provide a detector in the form of an array of photodiodes, where the number of photodiodes gives a certain detection resolution. An alternative technique employs a CMOS array.

Alternatively, the detector may be arranged to travel along a defined track, measuring light intensity at various points.

The signal recorded at particular locations along the detector 140 gives a measure of the degree of scatter experienced by the light from source 110. The degree of scatter is indicative of the size and concentration of particles in the effluent. In particular, larger particles tend to result in a larger displacement from the axis 111, and the intensity of light at a particular angle is indicative of the number of particles of a particular size. In the case of peritoneal dialysis, the particles of interest are leukocytes having a particular size. The number of such particles determined by measured light intensity at one or more given angular displacements may be compared to a defined threshold to indicate a potential issue for the user in question.

It has been found that the presence of leukocytes in the effluent causes scattering of the laser beam from source 110, away from axis 111. FIG. 3 shows a further illustration of the principle involved, whereby light from source 110 illuminates sample cuvette 120, including effluent. In the first embodiment, sample is in the form of a continuous flow and, in the second embodiment, the sample is static. The mode of operation of both embodiments is identical. The light is scattered by the presence of the leukocytes in the sample and causes the light to be scattered, as shown. The degree of scattering is recorded by the detector 140. The degree and intensity of scattering correlates with the concentration of leukocytes in the sample, as described previously.

It is found that in practice, the angle associated with a particular particle size depends on a number of factors including light intensity, the distance between the light source and the detector, path length through the cuvette amongst others. As such, the displacement angle associated with a particular particle size can vary depending upon the precise configuration employed.

It has been found that, in practice, the angular displacement tends to fall in the range of 0 to 15° from the central axis 111. In particular, the range 0 to 12° is found to offer the best range, since readings at displacements above this range tend to be too small to be distinguishable.

FIGS. 4a to 4c show details of a configuration of certain parts of the device according to an embodiment of the present invention.

In FIG. 4a, the optical source 110 is shown. It emits light towards and through cuvette 120, through a lens 112. The light thereby produced is scattered by the presence of leukocytes in the sample and so the light which leaves the cuvette is scattered somewhat. This scattered light is received at a detector 140. An individual photodetector is a photodiode 141, with a plurality of these forming the overall detector 140. Since each photodetector 141 occupies a finite space, it is difficult to locate many such photodetectors 141 in the space available. Since the scattering caused by the sample is essentially symmetrical about the central axis, on one side of the axis, there is provided a number of photodetectors associated with odd angles of scatter (1°, 3°, 5°, 7°, 9°, 11°) whilst on the other side of the axis, are a number of photodetectors associated with even angles of scatter (2°, 4°, 6°, 8°, 10°, 12°). In this way, more detectors may be provided in a given space, providing greater granularity in the results.

FIGS. 4b and 4c show, respectively, a top view looking down on the arrangement of FIG. 4a and a side view looking at the same arrangement. These figures are provided to illustrate that the light from the source 110 strikes the cuvette at a slight angle, in the region of 2°, which is sufficient to ensure that there is little or no reflection back into the source 120, as this could damage the source and/to adversely affect the results.

A clinical diagnosis of peritonitis is associated with a concentration of 10⁵ cells/ml. Embodiments of the present invention are able to detect a concentration of cells in the region of 10⁴ cells/ml. While such a concentration may be present in a healthy user, the ability to detect changes in the range 10⁴ to 10⁵ cells/ml, offers an opportunity to provide an early warning of infection.

FIG. 5a shows a particular form of cuvette 200, which may be employed in place of cuvette 120 shown previously, for use in the first (dynamic) embodiment. The cuvette 200 is connected at one end to a male connector 220 and at a second end to a female connector 230. A flow path 201 is defined within the cuvette in which the effluent or other sample fluid runs. Each of the male and female connectors is attached to the effluent line so that effluent runs through the cuvette 200 and into the reservoir 20.

The cuvette 200 is formed from an opaque plastics material, such that only light from laser or LED 110, which passes through window 210, enters the cuvette. To prevent or minimise unwanted light from entering the cuvette 200 along the flow path, first and second kinks or meanderings 202 are provided proximal each end of the flow path 201 in the cuvette. These have the effect of impeding stray light from the effluent line 21 from entering the cuvette 200 and possibly interfering with the analysis.

The kinks 202 also have the effect of inhibiting the production of bubbles in the flow path which could otherwise interfere with the analysis.

One surface of the cuvette 200 is transparent or less opaque and this is provided with an opaque sticker or seal 240 which is provided with a window or aperture 241 to facilitate the passage of light from laser or LED 110.

The cuvette 200 is intended to be disposable and used once only.

FIG. 5b shows a cuvette 250 for use in the static system, where there is no flow of effluent through the cuvette. Here, an effluent sample is introduced into the cuvette via cap 256 located at the top of the cuvette. This ensures that no sample can escape once introduced and the cap is closed. The cuvette 250 takes the form of a cartridge, for insertion into the diagnostic device. The majority of the cuvette is formed from an opaque plastics material so as to inhibit any stray light from entering the interior of the cuvette and to minimise any internal reflections. However, there are pairs of windows in the opaque plastic, which expose the substantially transparent inner container of the cuvette. The first pair of windows 254 are located towards the top of the cuvette and are provided to permit a simple visual inspection to ensure that sample is present and to an appropriate level. They serve no operational role beyond this.

The second pair of windows 252 are located towards the bottom of the cuvette and are arranged in the central optical axis whereby light from the source 110 passes through the rear and front windows 252 and then towards the detector. In FIG. 5b, only one of the pair of windows is visible, but there is a corresponding window on the opposite face of the cuvette 250. This pair of windows 252 are provided in a slightly recessed position in the cuvette. The size of the recess is intended to prevent inadvertent touching of the windows by the user, since grease or other materials on the user's fingers could adversely affect the operation of the device.

The cuvette 250 is provided with a relatively flared front surface 260, whereby the outer diameter of this surface is greater than the outer diameter of the opposing surface. This allows the cuvette to be “keyed” such that it can only be inserted into the diagnostic device in a single orientation. This is since the inner container of the cuvette, which contains the sample does not necessarily have symmetrical optical properties and should be used in a single orientation only, hence the “keyed” arrangement.

Also provided on the front surface of the cuvette is a label 258 which may comprise an identification symbol, such as a barcode or a QR code.

FIG. 6 shows a graph illustrating the effect of scattering of the light, from light source 110, through a sample in cases having different concentrations of leukocytes. The x-axis is a measure of the displacement (i.e. the angle of scatter, in degrees) from axis 111. In FIG. 6, this runs from 0° to 12°. The y-axis records the intensity of the signal provided by detector 140 and so is a measure of the intensity of light at a particular displacement.

In this particular example, a 5 mW Laser is used, having a wavelength in the region 650-700 nm. Different wavelengths and powers give different results, and the example given is only one of several options. The skilled person will realise that a different power will lead to different readings of light intensity and a different wavelength will lead to a different response. A wavelength in the range of 650-700 nm is found to yield good results in detecting and determining a concentration of leukocytes.

The curves shown represent five different concentrations of leukocytes: 5,000 cells/ml (300); 10,000 cells/ml (301); 25,000 cells/ml (302); 45,000 cells/ml (303); and 10,000,000 cells/ml (310).

As can be seen, the profile of the respective curve follows a regular trajectory from a low concentration (300) to a higher concentration (303). However, when the concentration tends much higher, towards 10⁷ cells/ml (310), the trajectory abruptly changes and the resultant profile ceases to resemble those corresponding to lower concentrations. Essentially, there comes a point where rather than present as a decaying curve (301-303), the curve has a much more linear profile where the gradient is substantially constant across the measured range.

It would be tempting, if one were considering just the first value at the 1° displacement, to think that that value alone is indicative of the concentration of leukocytes. That analysis is true up to a point, but as can be seen by curve 310, this breaks down at some point, when the curve changes form and profile considerably. The first value of curve 310 would appear to indicate a much lower concentration than is actually the case. As such, a more sophisticated approach is required. Such an approach requires more than a single result to be analysed.

In general, the more values of displacement that are analysed, the more accurate the response. At a minimum, two points require analysis. It is convenient to consider the two points closest to the central axis, namely 1° and 2°. It will be noted that the granularity adopted here is 1°, but different embodiments may use a smaller or larger increment and the results will need to be adjusted accordingly. However, the basic principle remains the same and it is instructive to consider the 1° increment set out here.

By examining the first two values of curve 300, it can be seen that the light intensity reading at 1° is a little over 2 and the reading at 2° is approximately 1. As such, it can be seen that the intensity has fallen by about 50% from 1° to 2°. By examining the curves 301 to 303, a similar change in amplitude is observed.

However, by examining the first two values of the curve 310, it can be seen that there is only a slight decrease from the reading at 1° to the reading at 2°. As such, there is clearly a different mechanism at work here and a different approach is required to calculate the associate leukocyte concentration. In this case, the GWR technique is used, as opposed to the AUC technique referred to earlier.

Between the concentration associated with the curve 303 (45,000 cells/ml) and the concentration associated with the curve 310 (10,000,000 cells/ml) the curve profile changes. In order to apply the correct technique, a threshold is determined which dictates which technique of the two is used. It has been found that once the second value (measured at 2°) is less than the first value (measured at 1°) by less than a 25% difference, then the GWR method should be used. If the difference is more than 25%, then the AUC method should be used.

In practice, the absolute value of the threshold may differ, depending upon the particular setup of the apparatus and a threshold value between 10% and 40% may be appropriate with 25% being a preferred value in a particular case only.

In order to account for different practical arrangements, it is instructive to specify the threshold in terms of a percentage decrease per degree of displacement. Hence, in the present example, the threshold may be considered as 25% per degree of displacement. In another embodiment, where intensity readings are taken at intervals of other than 1°, then the calculation may be adjusted accordingly.

Note also, that the best results are achieved proximal to the central axis 111.

An alternative and in, some ways, complementary, technique examines the gradients at two locations on each curve and by means of a comparison therebetween, the correct technique is selected.

Here, an embodiment of the invention is arranged to examine a gradient at one end of the range and compare this with a gradient at another end of the range. In the example shown, the gradient at a first end of the range may be determined by examining the readings from the photodetectors arranged at 1° and 2°, determining a gradient from these two results, and then repeating the process with the readings from the photodetectors arranged at 11° and 12°. In this case, gradient is defined as the difference between two readings, divided the angular displacement between the same readings. The units of intensity are not relevant to this calculation, since a comparison of numerical values is all that is required,

It can be seen that for curves 300-303, the first gradient is relatively steep and so indicative of a relatively lower concentration, whereas the second gradient is relatively flat, which acts to confirm this first approximation. However, for curve 310, associated with a much higher concentration, the first gradient is relatively flat, as is the second one, and so the similarity between them is indicative of this much higher concentration.

Depending on the result of the comparison between the first and second gradient, as set out above, the device is arranged to calculate the concentration of cells using one of two different techniques. If the first gradient is different to the second gradient by more than a defined threshold, then an “area under the curve”, AUC, technique is used, whereas if the first and second gradients are substantially the same, within a further defined range, then a “Generalised Weighted Ratio”, GWR, technique is used.

Calculating the AUC of the plot of scatter angle vs light intensity can be achieved using any convenient method such as the trapezoidal rule or other methods of integration. Within the appropriate range of cell concentrations, the cell concentration is proportional to the AUC and can be found by suitable calibration with known standards.

The GWR method uses the light intensity recorded at 4 or more scatter angles/displacements and calculates a weighted average intensity. The weighting given to each angle can be found by a number of empirical and computational methods, such as regression analysis, based on measurements of known standards. The weighted average of the light intensities can then be used to calculate the cell concentration in unknown samples within the applicable range of concentrations.

It has been found, empirically, that these two techniques offer better results in different situations, corresponding to different concentrations of leukocytes in the sample under analysis. If the concentration is relatively high, then the GWR method yields better results. However, if the concentration is relatively low, then the AUC method yields better results.

The comparison of gradients referred to above, which determines which method is to be used involves a switching point or threshold. For instance, if the first and second gradients do not differ by more than 25% then this is determined to be substantially the same for the purposes of determining the concentration and so the GWR method is used. However, if the first and second gradients differ by more than the same percentage, then this is determined to be substantially different and so the AUC method is used.

It can be seen from the above that a single reading at relatively small displacements may not be able to adequately distinguish between the different concentrations, and so it is necessary to examine the values recorded at multiple points, as exemplified by studying the first and second gradients.

In an enhanced embodiment, the profile across all photodetectors is compared with one or more pre-stored profiles to better match the observed result with pre-calculated or pre-modelled concentration values. By means of a form of pattern matching in this way, the prior techniques, based on analysing a plurality of points, are implicitly analysed and the result is still affected by these properties of the measured profile.

It is notable that the scattering observed and illustrated in FIG. 6 is due to cells of a particular size, which corresponds to the size of leukocytes. Particles of significantly different size will not affect the results. In particular, bacteria, which are significantly smaller, and which may be present in relatively high concentrations in a sample from a patient, do not adversely affect the ability of a device according to an embodiment of the invention to determine a concentration of leukocytes as set out above.

The device 100 may be provided with a display to indicate information to the user regarding the ongoing analysis. In a simple case, this could be in the form of an alert if the cell level is above a predefined threshold. More complex displays may return detailed results, although these may not be suitable or required in all cases.

In a further embodiment, the device 100 may be provided with a form of communication interface whereby the user's results are uploaded to a remote computer system, which may comprise the user's personal records and/or a remote monitoring station whereby problematic results may be reviewed by a physician who may contact the patient in case of concern for their wellbeing.

The communication interface may comprise a local wireless connection (such as Bluetooth) to a PC, which is then further connected via the internet to the remote computer system.

Alternatively, the device 100 may be able to communicate directly with the remote computer system via an integral cellular technology, for instance.

Embodiments of the present invention provide many advantages over prior art solutions. For instance, use of the device requires no specialist knowledge on the part of the user, who is able to use it as an integral part of their dialysis set up.

There is no chemistry involved in the process, meaning that there is no requirement to maintain a stock of indicators, reagents and the like.

The test is performed entirely in situ as part of the process it is monitoring. This means that there is no requirement to send samples to a lab for subsequent analysis.

Any issues which arise during dialysis are flagged instantly to the user and/or a physician (as required).

In the foregoing description, embodiments have been described in the context of peritoneal dialysis. However, other fluids may be analysed. These may include ascitic fluid, urine, sputum, pleural fluid and cerebral spinal fluid.

The foregoing embodiments are focussed on an embodiment of the invention primarily intended for use in a diagnostic setting, possibly in a clinic or a patient's home. However, a further embodiment which operates according to exactly the same principles may be used in a laboratory setting. There is often a need to determine a cell concentration in a sample and the aforementioned embodiment may be used in this way.

In a laboratory setting, the options available to a user may be slightly different to those presented to a patient, since the laboratory worker will typically have a higher skill level in the use of the equipment and may require a different level of detail to a patient. In the aforementioned embodiment, the apparatus is arranged to measure a concentration of leukocytes in particular. However, in a laboratory setting, the user may be interested in analysing other cell types. These may include, for instance, HL60 and Jurkat cells. Measurement of the concentration of these cell types may require an adjustment in the operation of the device, so that different result profiles will require a different processing to that applied to leukocytes. To facilitate this, an additional menu option may be provided so that the user is able to select the most appropriate processing to apply to the particular sample being analysed.

FIG. 7a shows, for completeness, a schematic representation of certain functional parts of the device 100 according to an embodiment of the present invention. The device 100 is controlled by processor 400. Processor 400 is any suitable microprocessor or microcontroller, which is programmed to perform the operations required in order to perform the functions set out above. The program which causes the processor 400 to operate is stored in memory 420. The same memory 420 can provide working memory as well as data, such as pre-stored profiles and thresholds used in the analysis of a sample.

The device 100 is operated via power supply 420. This may comprise an internal battery supply or an external mains-operated supply, as required.

The processor 400 is arranged to drive the optical source 110 and to receive signals from the optical detector 140, as set out above.

A result of the analysis is displayed on display 410. The same display may be configured to provide operational information, such as power status, as well as other warning or informational messages for a user.

FIG. 7b shows a representation of a device 100 according to an embodiment of the invention, for receiving an effluent tube 21, directly into the device, without the need for an additional cartridge or cassette as described previously.

The device 100 comprises two portions 100a, 100b, separated by a channel 101, which is recessed from an upper surface of portions 100a, 100b. The size of the channel 101 is selected such that it can receive a given diameter of effluent tube 21. The effluent tube 21 is generally formed from an elastomeric material and is pliable enough to be held snugly within the channel without the need for any further devices to secure it in place. Once positioned in the channel, the effluent tube 21 is able to cooperate with other elements of the device to enable the effluent flowing therein to be analysed.

By reference to FIGS. 8 and 9, a light source (e.g. a Laser) may be accommodated in portion 110b of the device 100 and a detector may be accommodated in portion 100a of the device 100. In this way, light from the Laser may pass through the tube 21 located in the channel 21, such that the detector may receive scattered light, so that analysis of the scattered light may provide the information required of certain material concentrations in the effluent.

FIGS. 8 and 9 illustrate alternative embodiments which each make use of the inherent optical properties of effluent tube 21 to assist in focussing light from laser 110 onto the detector array 140.

In FIG. 8, an embodiment is shown in a top view (a) looking down the effluent tube 21 and in side view (b). Here, light from laser 110 passes through plano-convex lens 114, which tends to cause the light beam to converge to a focus point and then diverge, until the rays strike the curved outer surface of tube 21, which tends to collimate the beams. The light then passes through the effluent sample in the tube, until the light beams are refocussed by the opposing side of the tube, such that the beams are then properly focussed on the detector array 140. The detector array, or row of individual detectors, runs parallel to the longitudinal direction of the tube 21.

The passage of light through the system can be seen clearly in FIG. 8(a) and (b). This system advantageously utilises the inherent properties of the tube 21 to assist in the detection process.

In FIG. 9, an embodiment is shown in a top view (a) looking down the effluent tube 21 and in side view (b). Here, light from laser 110 passes through plano-convex lens (or rectangular cylindrical lens) 114, which tends to cause the light beam to converge to a focus point which lies on a central longitudinal axis of the tube 21. In this way, the light always passes perpendicular to the walls of the tube 21. On exit from the tube 21, two Multiple-order Diffractive Engineered Surface (MODE) lenses 116 (or a single biconvex lens, not shown) focus light onto the detector array 140. The detector array, or row of individual detectors, runs parallel to the longitudinal direction of the tube 21.

The lenses 114, 116 are all identical and are typically rectangular cylindrical lenses 25 mm long, 12.5 mm wide, with a focal length of 20 mm. These are found to be suitable for an effluent tube having a diameter of in the range 4-10 mm, more preferably 6-8 mm. Other lens arrangements may be suitable. In essence, the lenses are arranged such that collimated light from the laser 110 is focused by lens 114 such that the collimated light from the laser focuses substantially in the middle of the tube 21 as shown in FIG. 9(a). The light than naturally diverges from that central point and is re-collimated by the lens arrangement 116 on the other side, before being focussed on the detector 140.

The passage of light through the system can be seen clearly in FIG. 9(a) and (b). This system advantageously utilises the inherent properties of the tube 21 to assist in the detection process. The inherent properties of the tube might simply be its transparent nature. In some circumstances, the curved profile of the tube can assist with the lensing effect which is provided by the lenses 114,116.

FIG. 10 shows a further embodiment whereby the effluent sample can be analysed directly in the effluent reservoir 20. Effluent reservoir 20 is a flexible, watertight, transparent bag arranged at the end of the effluent tube 21. It is provided to collect effluent discharged from the patient's abdomen.

A receiver 500 is provided, which includes a slot or other holder to receive the reservoir 20. The receiver 500 also includes the circuitry described previously which is provided and operable to analyse the effluent to assess a cell concentration.

The slot or holder is arranged such that the reservoir 20 is at least partially surrounded on a first and second, opposite, side. On a first surface of the slot or holder, there is provided the light source 110, as described previously. On an opposed surface of the slot or holder is the detector array 140. As can be seen in FIG. 10, the effluent reservoir 20 is arranged to sit between the light source 110 and detector array 140. The previously described operations are performed, such that the effluent in the reservoir 20 is analysed by virtue of the degree of scatter observed.

FIGS. 11 to 15 illustrate a still further embodiment which allows the effluent in the reservoir to be analysed directly and in-situ.

In this embodiment, a receiver 510 is provided which consists of a hinged clamshell arrangement with a base 520 arranged to receive the reservoir 20. Connected by a hinge 513, is lid 522, arranged to be closable and to secure the reservoir 20 in place. A lock mechanism 511, 512 is provide to secure the lid 522 in a closed position.

The base 520 comprises the detector array 140, arranged to sit under the effluent reservoir, once it is in place. The lid 522 comprises the light source 110, positioned to be opposite the detector array 140, with the reservoir 20 positioned therebetween, when the lid 522 is closed and locked in position,

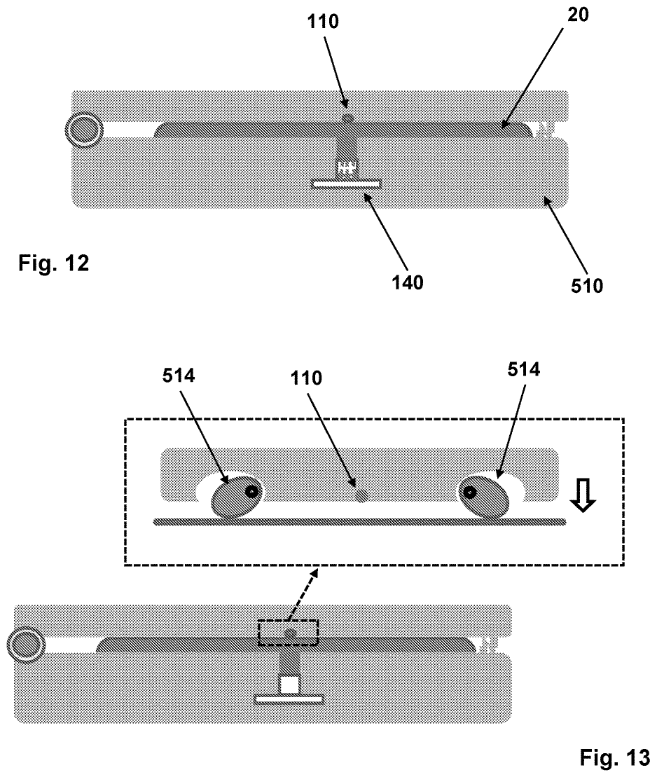

FIG. 11 shows the device 510 in an open configuration with the reservoir 20 loaded in position. FIG. 12 shows the lid 522 in the closed configuration. This also shows the alignment of the light source 110 and detector array 140. The device 510 can be switched into operational mode by detecting the engagement of the lock mechanism 511, 512. Additionally, or alternatively, one of more user interface elements, such as switches, buttons or other controls may be provided on the device 510.

FIG. 13 shows an expanded detail of an enhancement which ensures that the surface of the reservoir 20 is smooth and substantially planar, to ensure that a light path between the light source and the detector is arranged optimally.

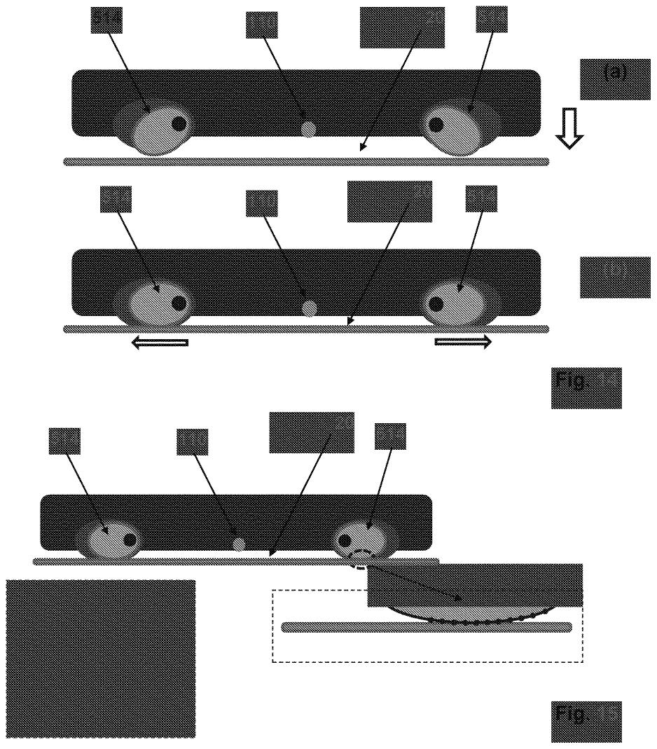

Positioned either side of the light source 110 in the lid 522, is an elliptical roller 514, which is arranged to partially rotate about a fixed point. The rollers 514 are biased to assume the slightly protruding configuration shown in FIG. 13 and in FIG. 14(a). When the lid 522 is closed, the rollers are pushed back into the body of the lid as shown in FIG. 14(b). Due to their elliptical nature and the axis of rotation being located off-centre, the rollers tend to stretch and flatted the immediate surface of the reservoir 20. This ensures that the point at which light from the light source 110 strikes the reservoir is smooth and behaves in an optically predictable manner.

FIG. 15 illustrates how the surface of the elliptical rollers 514 may be provided with relatively small projections or ridges. These, and/or the entire roller, may be formed from a suitable frictional material, such a rubber, silicone or the like, to enhance the grip of the rollers 514 on the surface of the reservoir 20.

FIG. 16 shows a so-called cassette 600 used in an Automated Peritoneal Dialysis system, The cassette 600 forms part of a system which controls the dialysis process. Cassettes typically consist of one or more pump chambers 620 used to move fluid around the system, one or more fluid inlets for dialysis fluid 640, a connection to the patient 630 used to instil dialysis fluid into the patient and drain spent fluid from the patient and a drain outlet 650 used to drain spent effluent from the system. Two separate areas 610 have been identified which provide optical access to the effluent in a manner which allows analysis to be performed as set out previously. Any suitable area in fluid connection with the drainage outlet 650 could be used.

The two areas 610 identified, in use, carry effluent, and are fundamentally accessible for analysis purposes.

FIG. 17 shows a Automated Peritoneal Dialysis machine 700, with the cassette 600 in position, ready to use. As can be seen, the cassette is positioned accurately and, in use, concealed behind a door 710. The device 700 is adapted such that the light source 110 and detector array 140 are provided, one on either side of the cassette, so that light can pass through the cassette from the light source 110 to the detector array 140, permitting the aforementioned analysis to occur. For instance, the light source 110 may be positioned in the door 710, with the detector in the body of the device 700 or vice-versa. Given the controlled nature of the fitting of the cassette 600, the accurate positioning of the light source 110 and detector array 140 is readily achieved to align with one of areas 610 in the cassette 600.

An advantage of such an arrangement is that it permits existing equipment to be utilised as far as possible and does not require any new or additional steps from a user of the device, who is already familiar with its operation.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A device for determining a concentration of cells of a given size in a sample of fluid, the sample comprising a plurality of cells, comprising:
a light source arranged to emit light along a central axis to illuminate the plurality of cells in the fluid sample;
a container to contain the sample;
a detector arranged to receive light at a plurality of displacements from the central axis, the light having passed through the plurality of cells in the fluid sample, and to produce a plurality of signals, each associated with, and indicative of an intensity of the received light at, a respective one of the plurality of displacements; and
a processor arranged to:
obtain a first light intensity reading at a first displacement from the central axis;
obtain a second light intensity reading at a second displacement from the central axis;
determine a difference between the first and second light intensity readings;
determine the concentration of cells based on the difference; wherein the container is one of:
an effluent line arranged to convey effluent from a patient;
an effluent bag arranged to collect effluent from a patient; and
a cassette forming part of an automated peritoneal dialysis system.

2. The device of claim 1 wherein if the container is the effluent line, optical properties of the effluent line are utilised in a light path from the light source to the detector.

3. The device of claim 2 wherein the optical properties of the effluent line include focussing or collimating.

4. The device of claim 1, wherein the container is an effluent line arranged to convey effluent from a patient, wherein collimated light from the light source is focused by a first lens, such that it focuses within the effluent line.

5. The device of claim 4 wherein the focused light diverges beyond the focal point and is then collimated and focussed on the detector by a second lens arrangement.

6. The device of claim 5 wherein the second lens arrangement comprises either:
a pair of lenses where the first of the pair acts to collimate and the second of the pair acts to focus the collimated light on the detector; or
a biconvex lens.

7. The device of claim 1, wherein the container is an effluent line arranged to convey effluent from a patient and the device comprises a receiver for receiving and temporarily retaining the effluent line in a configuration wherein the effluent line is accommodated between the light source and the detector.

8. The device of claim 7, wherein the receiver is a channel dimensioned to closely match the dimensions of the effluent line such that the effluent line is held securely in position inserting the effluent into the channel.

9. The device of claim 1, wherein the device is a diagnostic device.

10. The device of claim 1, wherein the detector comprises a plurality of photodetectors, each associated with one of the plurality of displacements.

11. The device of claim 10 wherein the plurality of photodetectors are arranged on both sides of the central axis, such that a first subset of the plurality of displacements is positioned on a first side of the central axis and a second, non-overlapping, subset of the plurality of displacements is positioned on a second side of the central axis.

12. The device of claim 1, wherein the light source is arranged to emit light of a single wavelength.

13. The device of claim 1, wherein the light source is a laser.

14. The device of claim 13 wherein the single wavelength of light is in the range of 650 nm to 700 nm.
